(19) United States
(12) Patent Application Publication
(10) Pub. No.: US 2008/0003196 A1
(43) Pub. Date: Jan. 3, 2008

(54) Absorbable cyanoacrylate compositions

(76) Inventors: Jerry Y. Jonn, Raleigh, NC (US); Ibraheem T. Badejo, Raleigh, NC (US); Julian A. Quintero, Raleigh, NC (US); Teresa Warren, Raleigh, NC (US); Melanie A. Vander Klok, Raleigh, NC (US)

Correspondence Address: HUTCHISON LAW GROUP PLLC, PO BOX 31686, RALEIGH, NC, 27612, US

(21) Appl. No.: 11/479,424

(22) Filed: Jun. 30, 2006

(51) Int. Cl. A61K 31/74

(52) U.S. Cl. 424/78.08

(57) ABSTRACT

An adhesive composition is provided including one or more polymerizable cyanoacrylate monomers and boron trifluoride as a stabilizer or complexing agent. The adhesive composition may also include or be used with a decomplexing agent, particularly one or more quaternary ammonium fluoride salts or one or more quaternary ammonium ether salts. A polymerization initiator or accelerator may also be used. The viscosity of the adhesive composition may be controlled by addition of a thickening agent which may be a polymer or copolymer catalyzed by a boron trifluoride complex or compound. Methods for the application of the adhesive compositions to living tissue are also provided.

### BACKGROUND

1. Field

The invention relates to stabilized monomer and absorbable polymer adhesive and sealant compositions, and to their use for industrial and medical applications.

2. State of the Art

Monomer and polymer adhesives are used in both industrial (including household) and medical applications. Included among these adhesives are the 1,1-disubstituted ethylene monomers and polymers, such as the α-cyanoacrylates. Since the discovery of the adhesive properties of such monomers and their resulting polymers, they have found wide use due to the speed with which they cure, the strength of the resulting bond formed, and their relative ease of use. These characteristics have made α-cyanoacrylate adhesives the primary choice for numerous applications such as bonding plastics, rubbers, glass, metals, wood, and, more recently, biological tissues.

Polymerizable 1,1-disubstituted ethylene monomers, and adhesive compositions comprising such monomers, are disclosed in U.S. Pat. No. 5,328,687 to Leung et al. Suitable methods for applying such compositions to substrates, and particularly in medical applications, are described in, for example, U.S. Pat. Nos. 5,928,611; 5,582,834; 5,575,997; and 5,624,669, all to Leung et al.

Medical applications of 1,1-disubstituted ethylene adhesive compositions include use as an alternate or an adjunct to surgical sutures and staples in wound closure as well as for covering and protecting surface wounds such as lacerations, abrasions, burns, stomatitis, sores, and other surface wounds. When an adhesive is applied, it is usually applied in its monomeric form, and the resultant polymerization gives rise to the desired adhesive bond.

However, at ordinary temperatures, the monomeric form may run when applied to surfaces. As a result, the monomeric adhesive may spread into a wound or along a surface to areas that do not require an adhesive. Therefore, the monomeric form must be controlled in order to prevent undue escape of the adhesive from any given area to which the adhesive is applied. Additionally, sufficient time must be allowed for the monomeric material to polymerize and thus to bring about the desired bonding action. In order to achieve a suitably viscous adhesive, thickening agents may be added to the monomer compositions.

For example, U.S. Pat. No. 3,527,841 to Wicker et al. discloses α-cyanoacrylate adhesive compositions for both general and surgical uses containing a viscosity modifier that is soluble, after heating, in a wide range of the esters of α-cyanoacrylic acid. The viscosity modifier is disclosed as poly(lactic acid).

U.S. Pat. No. 5,665,817 to Greff et al. discloses alkyl cyanoacrylate compositions suitable for topical application to human skin. The compositions may comprise a suitable amount of a thickening agent to provide a compositional viscosity suitable for certain applications onto human skin. The thickening agent is added to provide a viscosity of from about 2 to 50,000 centipoise at 20° C. The thickening agent employed is any biocompatible material that increases the viscosity of the alkyl cyanoacrylate composition and includes, by way of example, a partial polymer of the alkyl cyanoacrylate, polymethylmethacrylate (PMMA), or other preformed polymers soluble in the alkyl cyanoacrylate.

U.S. Pat. No. 5,328,687 to Leung et al. also discloses adhesive compositions that may be used for bonding tissue. Compositions comprising α-cyanoacrylate monomers are preferred. The compositions may further contain adjuvant substances such as thickening agents. Suitable disclosed thickeners include, for example, polycyanoacrylates, polylactic acid, polyglycolic acid, lactic-glycolic acid copolymers, polycaprolactone, lactic acid-caprolactone copolymers, poly-3-hydroxybutyric acid, polyorthoesters, polyalkyl acrylates, copolymers of alkylacrylate and vinyl acetate, polyalkyl methacrylates, and copolymers of alkyl methacrylates and butadiene.

U.S. Pat. No. 6,743,858, to Hickey et al., relates to sterilized cyanoacrylate solutions containing thickeners including, but not limited to, poly(2-ethylhexyl methacrylate), poly(2-ethylhexyl acrylate) and cellulose acetate butyrate. Suitable thickeners in Hickey et al. also include, for example, polycyanoacrylates, polyoxalates, lactic-glycolic acid copolymers, polycaprolactone, lactic acid-caprolactone copolymers, poly(caprolactone+DL-lactide+glycolide), polyorthoesters, polyalkyl acrylates, copolymers of alkylacrylate and vinyl acetate, polyalkyl methacrylates, and copolymers of alkyl methacrylates and butadiene.

Some monomeric α-cyanoacrylates are extremely reactive, polymerizing rapidly in the presence of even minute amounts of an initiator, including moisture present in the air or on moist surfaces such as animal tissue. Monomers of α-cyanoacrylates are anionically polymerizable or free radical polymerizable, or polymerizable by zwitterions or ion pairs to form polymers. Once polymerization has been initiated, the cure rate can be very rapid, depending on the choice of monomer. In addition to the cure rate, the shelf life of the monomers ensures usage at the desired time. Therefore, in order to obtain a monomeric α-cyanoacrylate composition with a suitable shelf-life, polymerization inhibitors such as anionic and free radical stabilizers are often added to the compositions. However, addition of certain stabilizers may result in substantial retardation of the cure rate of the composition.

Thus, a need exists for improved polymerizable cyanoacrylate monomeric adhesive compositions having an acceptable shelf life without affecting the performance and/or reactivity of the adhesive including its biocompatibility. In addition, a need exists for polymerizable cyanoacrylate monomeric adhesive compositions which have sufficient viscosity for the intended use.

### SUMMARY

An adhesive composition is provided comprising one or more polymerizable cyanoacrylate monomers, a complexing agent for the one or more polymerizable cyanoacrylate monomers comprising boron trifluoride, and a decomplexing agent comprising at least one quaternary ammonium fluoride salt, at least one quaternary ammonium ether salt, or mixtures thereof.

In one embodiment, an adhesive composition is provided comprising a first monomer species comprising an alkyl ester cyanoacrylate having the formula

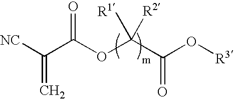

wherein R^{1′} and R^{2′} are, independently, H, a straight, branched or cyclic alkyl, or are combined together in a cyclic alkyl group, R^{3′} is a straight, branched or cyclic alkyl group, and m is 1-8; a second monomer species different from the first monomer species; a complexing agent for at least the first monomer species comprising boron trifluoride; a decomplexing agent comprising at least one quaternary ammonium fluoride salt, at least one quaternary ammonium ether salt, or mixtures thereof; and a polymerization initiator comprising at least one quaternary ammonium chloride salt.

In a further embodiment, an adhesive composition is provided comprising one or more polymerizable cyanoacrylate monomers, at least one anionic stabilizer, at least one free radical stabilizer, and at least one thickening agent comprising an absorbable polyester catalyzed by a boron trifluoride catalyst.

In a further embodiment, a method of treating living tissue is provided, comprising applying to living tissue a biocompatible adhesive composition comprising one or more polymerizable cyanoacrylate monomers and a complexing agent for the one or more polymerizable cyanoacrylate monomers comprising boron trifluoride, wherein the biocompatible adhesive composition is applied in conjunction with a decomplexing agent comprising at least one quaternary ammonium fluoride salt, at least one quaternary ammonium ether salt, or mixtures thereof.

### DETAILED DESCRIPTION OF THE PREFERRED EMBODIMENTS

A cyanoacrylate adhesive composition comprising one or more polymerizable cyanoacrylate monomers and a method of using such an adhesive composition is provided. Stabilization and polymerization of the monomeric cyanoacrylate adhesive composition is achieved through the addition of a stabilizer or complexing agent comprising a Lewis acid salt such as boron trifluoride to a polymerizable monomeric cyanoacrylate composition and utilization of a decomplexing agent comprising at least one quaternary ammonium salt to promote acceleration or initiation of the polymerization of the polymerizable monomeric composition, resulting in a cyanoacrylate adhesive composition.

The addition of a Lewis acid such as boron trifluoride to a polymerizable monomeric cyanoacrylate composition has been found to inhibit the reactivity of the monomeric cyanoacrylate composition, thus providing improved or increased stability to the polymerizable monomeric composition. The use of boron trifluoride, thus, increases and/or improves the shelf-life of the polymerizable monomeric composition.

In some instances, however, depending on the particular monomers used, for example, the addition of boron trifluoride to a polymerizable monomeric cyanoacrylate composition has been found to result in a reduction of reactivity of the polymerizable monomeric composition, sometimes resulting in a complete lack of polymerization of the cyanoacrylate monomer or monomers even after initiation of polymerization. It has now been found that boron trifluoride may be effectively used as a stabilizer or complexing agent in polymerizable monomeric cyanoacrylate compositions if at least one quaternary ammonium salt, such as a quaternary ammonium fluoride salt or a quaternary ammonium ether salt, is used as a decomplexing agent to promote acceleration or initiation of polymerization.

Without being bound to any theory, it is believed that the boron trifluoride may form a complex or reaction product with the one or more cyanoacrylate monomers, resulting in prevention, reduction or inhibition of polymerization of the monomer or monomers, hence stabilizing the monomeric composition. The BF₃ thus may serve as a complexing agent for the one or more cyanoacrylate monomers. The quaternary ammonium salt decomplexing agent is believed to remove the boron trifluoride from the complex or reaction product with the one or more polymerizable cyanoacrylate monomers, thus serving as a decomplexing agent, allowing the polymerization reaction of the monomers to proceed. Thus, the polymerization of the monomeric composition may be controlled by contacting a polymerizable monomeric cyanoacrylate composition stabilized or complexed with BF₃ with at least one quaternary ammonium salt before or at the time polymerization is desired. The quaternary ammonium salt preferably is a quaternary ammonium fluoride salt or a quaternary ammonium ether salt.

A stable cyanoacrylate adhesive composition comprising one or more polymerizable cyanoacrylate monomers is prepared by adding boron trifluoride to the monomer formulation. The boron trifluoride may be used in any form such as in a complex such as boron trifluoride diethyl etherate or in the vapor phase.

The polymerizable monomeric cyanoacrylate composition stabilized with BF₃ may be used safely in medical applications involving contact with living patients, including human patients. Moreover, the boron trifluoride stabilizer inhibits polymerization of the polymerizable monomer or monomers of the composition sufficiently to enable an acceptable shelf life for the stable monomeric cyanoacrylate composition. The stable polymerizable monomeric cyanoacrylate compositions are typically sterilized for use in medical applications. The stable polymerizable monomeric cyanoacrylate compositions may be sterilized by dry heat sterilization while retaining suitability for medical applications.

The boron trifluoride typically is used in the cyanoacrylate adhesive compositions comprising one or more polymerizable cyanoacrylate monomers in a stabilization effective amount. For purposes herein, a “stabilization effective amount” is an amount sufficient to provide at least partial stabilization or complexation of the polymerization monomer.

“Stabilization” or “stabilized” as used herein may be measured by the viscosity of the cyanoacrylate adhesive composition comprising one or more polymerizable cyanoacrylate monomers over a period of time since an indication of premature polymerization in cyanoacrylate monomer compositions is an increase in viscosity of the composition over time. That is, as the adhesive composition comprising one or more cyanoacrylate monomers polymerizes, the viscosity of the composition increases. If the viscosity becomes too high, i.e., too much premature polymerization has occurred, the composition becomes unsuitable for its intended use or becomes very difficult to apply. Thus, while some polymerization or thickening of the monomeric composition may occur, such as can be measured by changes in viscosity of the composition, such change, when the cyanoacrylate monomer composition is stabilized, is not so extensive as to destroy or significantly impair the usefulness of the compositions.

The boron trifluoride may be used in a stabilization effective amount which may be affected by the cyanoacrylate monomer or monomers used. By way of example, use of a certain amount of boron trifluoride may prevent entirely the polymerization of a particular alkyl ester cyanoacrylate monomer. In such cases, less boron trifluoride may be desirable for use while still providing a stabilization effective amount. In embodiments, the boron trifluoride may be used in an amount from about 1 to about 200 ppm, preferably, from about 20 to about 160 ppm.

In embodiments, other stabilizing agents may also be used in addition to the complexing agent boron trifluoride. Suitable free radical stabilizing agents for use in polymerizable cyanoacrylate adhesive compositions comprising one or more polymerizable cyanoacrylate monomers include hydroquinone, hydroquinone monomethyl ether, catechol, pyrogallol, benzoquinone, 2-hydroxybenzoquinone, p-methoxy phenol, t-butyl catechol, butylated hydroxy anisole, butylated hydroxy toluene, and t-butyl hydroquinone and mixtures or combinations thereof. The free radical stabilizing agents may be used in amounts from about 5 to about 10,000 ppm. In embodiments, if hydroquinone is used, the amount may be from about 5 to about 70 ppm and may be used in conjunction with butylated hydroxy anisole in an amount of about 500 to about 10,000 ppm.

The cyanoacrylate adhesive compositions comprising one or more polymerizable cyanoacrylate monomers may also optionally include both at least one anionic vapor phase stabilizer and at least one anionic liquid phase stabilizer. These stabilizing agents inhibit polymerization. Examples of such anionic agents are described for example, in U.S. Pat. No. 6,620,846, incorporated herein by reference in its entirety.

The anionic vapor phase stabilizers may be selected from among known stabilizers, including, but not limited to, sulfur dioxide or hydrogen fluoride. The amount of anionic vapor phase stabilizer that is added to the monomer composition depends on the identity of the liquid phase stabilizer(s) chosen in combination with it, the monomer to be stabilized, as well as the packaging material to be used for the composition. Typically, each anionic vapor phase stabilizer is added to give a concentration of less than about 200 parts per million (ppm). In embodiments, each anionic vapor phase stabilizer is present in an amount from about 1 to about 200 ppm, preferably from about 10 to about 75 ppm, even more preferably from about 10 to about 50 ppm, and most preferably from about 10 to about 20 ppm. The amount to be used can be determined by one of ordinary skill in the art using known techniques without undue experimentation.

In embodiments, the liquid phase anionic stabilizer is a very strong acid. As used herein, a very strong acid is an acid that has an aqueous pKₐ of less than 1.0. Suitable very strong acidic stabilizing agents include, but are not limited to, very strong mineral and/or oxygenated acids. Examples of such very strong acids include, but are not limited to, sulfuric acid (pKₐ −3.0), perchloric acid (pKₐ −5), hydrochloric acid (pKₐ −7.0), hydrobromic acid (pKₐ −9), fluorosulfonic acid (pKₐ<−10), chlorosulfonic acid (pKₐ −10). In embodiments, the very strong acid liquid phase anionic stabilizer is added to give a final concentration of about 1 to about 200 ppm. The very strong acid liquid phase anionic stabilizer may be present in a concentration of from about 5 to about 80 ppm, preferably from about 10 to about 40 ppm. The amount of very strong acid liquid phase anionic stabilizer to be used can be determined by one of ordinary skill in the art without undue experimentation.

In embodiments, the very strong acid liquid phase anionic stabilizer is sulfuric acid, perchloric acid, or chlorosulfonic acid.

The composition may also optionally include at least one other anionic stabilizing agent that inhibits polymerization. These agents are herein referred to as secondary anionic active agents to contrast them with the strong or very strong liquid phase anionic stabilizers, which are referred to hereinbelow as “primary” anionic stabilizers. The secondary anionic active agents can be included in the compositions to adjust the cure speed of the adhesive composition, for example.

The secondary anionic active agent would normally be an acid with a higher pKₐ than the primary anionic stabilizing agent and may be provided to more precisely control the cure speed and stability of the adhesive, as well as the molecular weight of the cured adhesive. Any mixture of primary anionic stabilizers and secondary active agents may be included as long as the chemistry of the composition is not compromised and the mixture does not significantly inhibit the desired polymerization rate of the composition. Furthermore, the mixture should not, in medical adhesive compositions, show unacceptable levels of toxicity.

Suitable secondary anionic active agents include those having aqueous pKₐ ionization constants ranging from 2 to 8, preferably from 2 to 6, and most preferably from 2 to 5. Examples of such suitable secondary anionic stabilizing agents include, but are not limited to, organic acids, such as acetic acid (pKₐ 4.8), benzoic acid (pKₐ 4.2), chloroacetic acid (pKₐ 2.9), cyanoacetic acid, and mixtures thereof. These secondary anionic stabilizing agents may be organic acids, such as acetic acid or benzoic acid. In embodiments, the amount of acetic acid and/or benzoic acid is about 25 to about 500 ppm. The concentration of acetic acid is typically about 50 to about 400 ppm, preferably about 75 to about 300 ppm, and more preferably about 100 to about 200 ppm.

The anionic stabilizers are chosen in conjunction such that the stabilizers are compatible with the chosen adhesive composition including the boron trifluoride complexing agent and each other stabilizer, as well as with the packaging material and the equipment used to make and package the composition. In other words, the combination of vapor phase stabilizer(s), liquid phase stabilizer(s), and monomer should be such that a stabilized, substantially unpolymerized adhesive composition is present after packaging (and sterilization, where the composition is intended for medical applications).

Cyanoacrylate adhesive monomer compositions including the stabilizers as described, and polymers formed therefrom, are useful as tissue adhesives, sealants for preventing bleeding or for covering open wounds, and in other biomedical applications. The monomer compositions find uses in, for example, preventing body fluid leakage, sealing air leakage in the body, tissue approximation, apposing surgically incised or traumatically lacerated tissues; retarding blood flow from wounds; drug delivery; dressing burns; dressing skin or other superficial or deep tissue surface wounds (such as abrasions, chaffed or raw skin, and/or stomatitis); and aiding repair and regrowth of living tissue. Monomer compositions of the present invention, and polymers formed therefrom, have broad application for sealing wounds in various living tissue, internal organs and blood vessels, and can be applied, for example, on the interior or exterior of blood vessels and various organs or tissues. Monomer compositions of the present invention, and polymers formed therefrom, are also useful in industrial and home applications, for example in bonding rubbers, plastics, wood, composites, fabrics, and other natural and synthetic materials.

Monomers that may be used in this invention are readily polymerizable, e.g. anionically polymerizable or free radical polymerizable, or polymerizable by zwitterions or ion pairs to form polymers. Some such monomers are disclosed in, for example, U.S. Pat. No. 5,328,687 to Leung, et al., which is hereby incorporated by reference in its entirety herein. Preferably, the cyanoacrylate adhesive compositions comprise one or more polymerizable cyanoacrylate monomers and are biocompatible. The cyanoacrylate adhesive compositions comprising one or more polymerizable cyanoacrylate monomers may include combinations or mixtures of cyanoacrylate monomers.

The term “biocompatible” refers to a material being suited for and meeting the requirements of a medical device, used for either long or short term implants or for non-implantable applications, such that when implanted or applied in an intended location, the material serves the intended function for the required amount of time without causing an unacceptable response. Long term implants are defined as items implanted for more than 30 days.

By way of example, useful monomers include α-cyanoacrylates of formula (I). These monomers are known in the art and have the formula

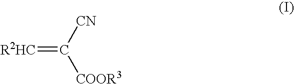

wherein R² is hydrogen and R³ is a hydrocarbyl or substituted hydrocarbyl group; a group having the formula —R⁴—O—R⁵—O—R⁶, wherein R⁴ is a 1,2-alkylene group having 2-4 carbon atoms, R⁵ is an alkylene group having 1-4 carbon atoms, and R⁶ is an alkyl group having 1-6 carbon atoms; or a group having the formula

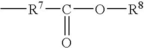

wherein R⁷ is

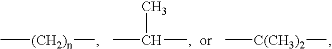

wherein n is 1-10, preferably 1-5 carbon atoms, and R⁸ is an organic moiety.

Examples of suitable hydrocarbyl and substituted hydrocarbyl groups include straight chain or branched chain alkyl groups having 1-16 carbon atoms; straight chain or branched chain C₁-C₁₆ alkyl groups substituted with an acyloxy group, a haloalkyl group, an alkoxy group, a halogen atom, a cyano group, or a haloalkyl group; straight chain or branched chain alkenyl groups having 2 to 16 carbon atoms; straight chain or branched chain alkynyl groups having 2 to 12 carbon atoms; cycloalkyl groups; aralkyl groups; alkylaryl groups; and aryl groups.

The organic moiety R⁸ may be substituted or unsubstituted and may be straight chain, branched or cyclic, saturated, unsaturated or aromatic. Examples of such organic moieties include C₁-C₈ alkyl moieties, C₂-C₈ alkenyl moieties, C₂-C₈ alkynyl moieties, C₃-C₁₂ cycloaliphatic moieties, aryl moieties such as phenyl and substituted phenyl and aralkyl moieties such as benzyl, methylbenzyl, and phenylethyl. Other organic moieties include substituted hydrocarbon moieties, such as halo (e.g., chloro-, fluoro- and bromo-substituted hydrocarbons) and oxy-substituted hydrocarbon (e.g., alkoxy substituted hydrocarbons) moieties. Preferred organic radicals are alkyl, alkenyl, and alkynyl moieties having from 1 to about 8 carbon atoms, and halo-substituted derivatives thereof. Particularly preferred are alkyl moieties of 4 to 6 carbon atoms.

In the cyanoacrylate monomer of formula (I), R³ may be an alkyl group having 1-10 carbon atoms or a group having the formula -AOR⁹, wherein A is a divalent straight or branched chain alkylene or oxyalkylene moiety having 2-8 carbon atoms, and R⁹ is a straight or branched alkyl moiety having 1-8 carbon atoms.

Examples of groups represented by the formula -AOR⁹ include 1-methoxy-2-propyl, 2-butoxy ethyl, isopropoxy ethyl, 2-methoxy ethyl, and 2-ethoxy ethyl.

The α-cyanoacrylates of formula (I) can be prepared according to methods known in the art. U.S. Pat. Nos. 2,721,858 and 3,254,111, each of which is hereby incorporated in its entirety by reference, disclose methods for preparing α-cyanoacrylates. For example, the α-cyanoacrylates can be prepared by reacting an alkyl cyanoacetate with formaldehyde in a nonaqueous organic solvent and in the presence of a basic catalyst, followed by pyrolysis of the anhydrous intermediate polymer in the presence of a polymerization inhibitor.

The α-cyanoacrylates of formula (I) wherein R³ is a group having the formula R⁴—O—R⁵—O—R⁶ can be prepared according to the method disclosed in U.S. Pat. No. 4,364,876 to Kimura et al., which is hereby incorporated in its entirety by reference. In the Kimura et al. method, the α-cyanoacrylates are prepared by producing a cyanoacetate by esterifying cyanoacetic acid with an alcohol or by transesterifying an alkyl cyanoacetate and an alcohol; condensing the cyanoacetate and formaldehyde or para-formaldehyde in the presence of a catalyst at a molar ratio of 0.5-1.5:1, preferably 0.8-1.2:1, to obtain a condensate; depolymerizing the condensation reaction mixture either directly or after removal of the condensation catalyst to yield crude cyanoacrylate; and distilling the crude cyanoacrylate to form a high purity cyanoacrylate.

The α-cyanoacrylates of formula (I) wherein R³ is a group having the formula

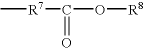

can be prepared according to the procedure described in U.S. Pat. No. 3,995,641 to Kronenthal et al., which is hereby incorporated in its entirety by reference. In the Kronenthal et al. method, such α-cyanoacrylate monomers are prepared by reacting an alkyl ester of an α-cyanoacrylic acid with a cyclic 1,3-diene to form a Diels-Alder adduct which is then subjected to alkaline hydrolysis followed by acidification to form the corresponding α-cyanoacrylic acid adduct. The α-cyanoacrylic acid adduct is preferably esterified by an alkyl bromoacetate to yield the corresponding carbalkoxymethyl α-cyanoacrylate adduct. Alternatively, the α-cyanoacrylic acid adduct may be converted to the α-cyanoacrylyl halide adduct by reaction with thionyl chloride. The α-cyanoacrylyl halide adduct is then reacted with an alkyl hydroxyacetate or a methyl substituted alkyl hydroxyacetate to yield the corresponding carbalkoxymethyl α-cyanoacrylate adduct or carbalkoxy alkyl α-cyanoacrylate adduct, respectively. The cyclic 1,3-diene blocking group is finally removed and the carbalkoxy methyl α-cyanoacrylate adduct or the carbalkoxy alkyl α-cyanoacrylate adduct is converted into the corresponding carbalkoxy alkyl α-cyanoacrylate by heating the adduct in the presence of a slight deficit of maleic anhydride.

Examples of monomers of formula (I) include cyanopentadienoates and α-cyanoacrylates of the formula:

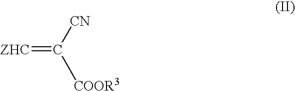

wherein Z is —CH═CH₂ and R³ is as defined above. The monomers of formula (II) wherein R³ is an alkyl group of 1-10 carbon atoms, i.e., the 2-cyanopenta-2,4-dienoic acid esters, can be prepared by reacting an appropriate 2-cyanoacetate with acrolein in the presence of a catalyst such as zinc chloride. This method of preparing 2-cyanopenta-2,4-dienoic acid esters is disclosed, for example, in U.S. Pat. No. 3,554,990, which is hereby incorporated in its entirety by reference.

Suitable α-cyanoacrylate monomers which may be used, alone or in combination, include alkyl α-cyanoacrylates such as 2-octyl cyanoacrylate; dodecyl cyanoacrylate; 2-ethylhexyl cyanoacrylate; butyl cyanoacrylate such as n-butyl cyanoacrylate; ethyl cyanoacrylate; methyl cyanoacrylate or other α-cyanoacrylate monomers such as methoxyethyl cyanoacrylate; 2-ethoxyethyl cyanoacrylate; 3-methoxybutyl cyanoacrylate; 2-butoxyethyl cyanoacrylate; 2-isopropoxyethyl cyanoacrylate; and 1-methoxy-2-propyl cyanoacrylate. In embodiments, the monomers are ethyl, n-butyl, or 2-octyl α-cyanoacrylate.

Other cyanoacrylates which may be used include alkyl ester cyanoacrylates. Besides the process detailed above, alkyl ester cyanoacrylates can also be prepared through the Knoevenagel reaction of an alkyl cyanoacetate, or an alkyl ester cyanoacetate, with paraformaldehyde. This leads to a cyanoacrylate oligomer. Subsequent thermal cracking of the oligomer results in the formation of a cyanoacrylate monomer. After further distillation, a cyanoacrylate monomer with high purity (greater than 95.0%, preferably greater than 99.0%, and more preferably greater than 99.8%), may be obtained.

Monomers prepared with low moisture content and essentially free of impurities (e.g., surgical grade) are preferred for biomedical use. Monomers utilized for industrial purposes need not be as pure.

In some embodiments, the alkyl ester cyanoacrylate monomers may have the formula:

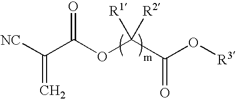

wherein R^{1′} and R^{2′} are, independently, H, a straight, branched or cyclic alkyl, or are combined together in a cyclic alkyl group, R^{3′} is a straight, branched or cyclic alkyl group, and m is 1-8. Preferably, R^{1′} is H or a C₁, C₂ or C₃ alkyl group, such as methyl or ethyl; R^{2′} is H or a C₁, C₂ or C₃ alkyl group, such as methyl or ethyl; R^{3′} is a C₁-C₁₆ alkyl group, more preferably a C₁-C₁₀ alkyl group, such as methyl, ethyl, propyl, isopropyl, butyl, isobutyl, pentyl, hexyl, heptyl, octyl, nonyl or decyl, and even more preferably a C₂, C₃ or C₄ alkyl group, and m is preferably 1-4.

Examples of the alkyl ester monomers may include, but are not limited to:

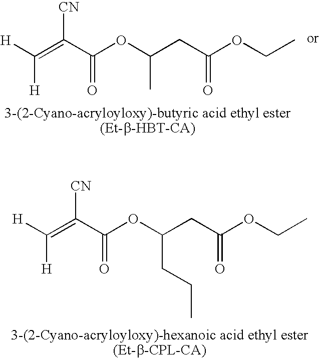

Additional examples of alkyl ester cyanoacrylates include, but are not limited to, butyl lactoyl cyanoacrylate (BLCA), butyl glycoloyl cyanoacrylate (BGCA), isopropyl glycoloyl cyanoacrylate (IPGCA), ethyl lactoyl cyanoacrylate (ELCA), and ethyl glycoloyl cyanoacrylate (EGCA) and combinations thereof. BLCA may be represented by the formula above, wherein R^{1′} is H, R^{2′} is methyl and R^{3′} is butyl. BGCA may be represented by the formula above, wherein R^{1′} is H, R^{2′} is H and R^{3′} is butyl. IPGCA may be represented by the formula above, wherein R^{1′} is H, R^{2′} is H and R^{3′} is isopropyl. ELCA may be represented by the formula above, wherein R^{1′} is H, R^{2′} is methyl and R^{3′} is ethyl. EGCA may be represented by the formula above, wherein R^{1′} is H, R^{2′} is H and R^{3′} is ethyl.

Other examples of alkyl ester cyanoacrylates include alkyl alpha-cyanoacryloyl caprolactate and alkyl alpha-cyanoacryloyl butrylactate. Other cyanoacrylates useful in the present invention are disclosed in U.S. Pat. No. 3,995,641 to Kronenthal et al., the entire disclosure of which is hereby incorporated by reference.

Alternatively, or in addition, alkyl ether cyanoacrylate monomers may be used. Alkyl ethyl cyanoacrylates have the general formula:

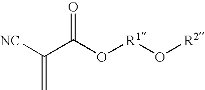

wherein R^{1″} is a straight, branched or cyclic alkyl, and R^{2″} is a straight, branched or cyclic alkyl group. Preferably, R^{1″} is a C₁, C₂ or C₃ alkyl group, such as methyl or ethyl; and R^{2″} is a C₁-C₁₆ alkyl group, more preferably a C₁-C₁₀ alkyl group, such as methyl, ethyl, propyl, isopropyl, butyl, isobutyl, pentyl, hexyl, heptyl, octyl, nonyl or decyl, and even more preferably a C₂, C₃ or C₄ alkyl group.

Examples of alkyl ether cyanoacrylates include, but are not limited to, isopropyoxy ethyl cyanoacrylate (IPECA) and methoxy butyl cyanoacrylate (MBCA) or combinations thereof. IPECA may be represented by the formula above, wherein R^{1″} is ethylene and R^{2″} is isopropyl. MBCA may be represented by the formula above, wherein R^{1″} is n-butylene and R^{2″} is methyl.

Alkyl ester cyanoacrylates and alkyl ether cyanoacrylates are particularly useful for medical applications because of their absorbability by living tissue and associated fluids. The terms “absorbable” or “absorbable adhesive” or variations thereof mean the ability of a tissue-compatible material to degrade or biodegrade at some time after implantation into products that are eliminated from the body or metabolized therein. Thus, as used herein, absorbability means that the polymerized adhesive is capable of being absorbed, either fully or partially, by tissue after application of the adhesive.

Likewise, the terms “non-absorbable” or “non-absorbable adhesive” or variations thereof mean completely or substantially incapable of being absorbed, either fully or partially, by tissue after application of the adhesive. Furthermore, relative terms such as “faster absorbing” and “slower absorbing” are used relative to two monomer species to indicate that a polymer produced from one monomer species is absorbed faster (or slower) than a polymer formed from the other monomer species.

For the purposes of this invention, the term “substantially absorbed” means at least 90% absorbed within about three years. Likewise, the term “substantially non-absorbed” means at most 20% absorbed within about three years. Preferably, 100% of the polymerized and applied cyanoacrylate when using these types of cyanoacrylate monomers may be absorbed in a period of less than 3 years, preferably approximately 2-24 months, more preferably 3-18 months, and most preferably 6-12 months after application of the adhesive to living tissue. The absorption time may vary depending on the particular uses and tissues involved. Thus, for example longer absorption time may be desired where the adhesive composition is applied to hard tissues, such as bone, but a faster absorption time may be desired where the adhesive composition is applied to softer tissues.

The selection of monomer will affect the absorption rate of the resultant polymer, as well as the polymerization rate of the monomer. Two or more different monomers that have varied absorption and/or polymerization rates may be used in combination to give a greater degree of control over the absorption rate of the resultant polymer, as well as the polymerization rate of the monomer.

According to some embodiments, the adhesive composition comprises a mixture of monomer species with varying absorption rates. Where two monomer species having different absorption rates are used, it is preferred that the absorption rates be sufficiently different that a mixture of the two monomers can yield a third absorption rate that is effectively different from the absorption rates of the two monomers individually. Compositions according to these embodiments are described, for example, in U.S. patent application Ser. No. 09/919,877, filed Aug. 2, 2001, published as U.S. Patent Publication No. 2002/0037310 on Mar. 28, 2002, and U.S. Pat. No. 6,620,846, both incorporated herein by reference in their entireties.

Absorbable cyanoacrylates have broad application for closure and hemostatic sealing of wounds and the like in various living tissue, including but not limited to internal organs and blood vessels. These absorbable formulations can be applied on the interior or exterior of various organs and tissues.

Adhesives as disclosed are biocompatible and may be applied internally or externally in or on living tissue.

For example, suitable compositions according to embodiments can be prepared by mixing suitable quantities of an alkyl alpha cyanoacrylate such as 2-octyl alpha-cyanoacrylate with one of butyl lactoyl cyanoacrylate (BLCA), butyl glycoloyl cyanoacrylate (BGCA), isopropyl glycoloyl cyanoacrylate (IPGCA), ethyl lactoyl cyanoacrylate (ELCA), and ethyl glycoloyl cyanoacrylate (EGCA). Such mixtures may range from ratios of about 90:10 to about 10:90 by weight, preferably about 75:25 to about 25:75 by weight such as from about 60:40 to about 40:60 by weight.

Some alkyl ester cyanoacrylate monomers may react slowly due to bulky alkyl groups, apparently limiting their applicability as surgical adhesives. By themselves, some alkyl ester cyanoacrylates cure in several hours, or in some cases do not fully cure at all. The use of boron trifluoride, although advantageous for providing stability to a monomeric cyanoacrylate adhesive composition, may slow reaction time or polymerization time further, particularly when used with such alkyl ester cyanoacrylates.

To overcome problems associated with slow polymerization of the monomers, a compatible agent which promotes initiation or acceleration of polymerization of the alkyl ester cyanoacrylate monomer or other cyanoacrylate monomer, may be used with the monomer composition. It has been discovered that the reduction in reactivity found with the use of boron trifluoride as a stabilization agent or complexing agent, believed to be due to the formation of a complex of the cyanoacrylate monomer or monomers with BF₃, may be alleviated or removed for polymerization by the addition of quaternary ammonium fluoride or quaternary ammonium ether salts as decomplexing agents which may serve to promote acceleration or initiation for the polymerizable monomeric cyanoacrylate composition.

The term “decomplexing agent” is used herein to include agents which decomplex a BF₃-cyanoacrylate monomer complex or reaction product which may be present and which may accelerate and/or initiate polymerization of the one or more cyanoacrylate monomers. Fluoride or ether salts of quaternary amines may be used alone or in combinations or mixtures as the decomplexing agent.

Alkyl ester cyanoacrylates, for example, stimulated to cure by a suitable quaternary ammonium fluoride salt or quaternary ammonium ether salt decomplexing agent may be made to cure in as short as a few seconds to a few minutes. The cure rate may be closely controlled by selection of an amount or concentration of decomplexing agent added to the composition comprising polymerizable cyanoacrylate monomer or monomers and may thus be readily controlled by one skilled in the art in light of the present disclosure. A suitable quaternary ammonium fluoride or ether salt decomplexing agent provides a consistent controllable complete polymerization of the monomer or monomers so that the polymerization of the monomer or monomers can be made to occur in the time desired for the particular application.

The quaternary ammonium fluoride salt or quaternary ammonium ether salt decomplexing agent which may be used in conjunction with BF₃ as the complexing agent for cyanoacrylate monomer(s) may be any of a group of ammonium salts in which organic radicals have been substituted for all four hydrogens of the original ammonium cation. As used herein, the quaternary ammonium fluoride salt will have the general formula A:

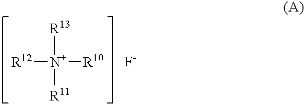

wherein R¹⁰, R¹¹, R¹² and R¹³ are each, independently, a substituted or unsubstituted straight, branched or cyclic alkyl group; a substituted or unsubstituted aromatic ring; or a substituted or unsubstituted aralkyl group, wherein the alkyl groups, aromatic rings or aralkyl groups may optionally further contain heteroatoms such as O and S. In embodiments, R¹⁰, R¹¹, R¹² and R¹³ are C₁-C₈ alkyl groups, preferably, C₁-C₄ alkyl groups, or an aralkyl group. By way of example, quaternary ammonium fluoride salts useful as decomplexing agents may include, but are not limited by, tetrabutylammonium fluoride, tetramethylammonium fluoride, tetraethylammonium fluoride, tetraoctylammonium fluoride, benzyltrimethyl ammonium fluoride or a combination thereof.

The amount of quaternary ammonium fluoride salt to be used as a decomplexing agent for and with the polymerizable monomeric cyanoacrylate adhesive compositions typically may depend on the amount of boron trifluoride present in the monomeric cyanoacrylate composition, the type or types of polymerizable cyanoacrylate monomers present and the desired rate of polymerization. Typically, the quaternary ammonium fluoride salt will be present in an amount of from about 10 ppm to about 10,000 ppm, preferably about 500 ppm to about 8000 ppm, more preferably about 600 ppm to about 7500 ppm.

As used herein, the quaternary ammonium ether salts will have the general formula B:

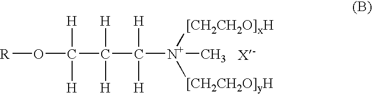

where R is a straight or branched alkyl group of from about 2 to about 20 carbon atoms, preferably from about 4 to about 16 carbon atoms; x and y represent the number of repeating units and independently are integers of from 1 to about 10, preferably from 1 to about 3, 4, or 5; and X′ is a counterion selected from, for example, halides such as chloride, bromide, iodide, and fluoride, sulfate, hydrogen sulfate, sulfite, hydrogen sulfite, bisulfate, bisulfite, hydroxide, and the like. Preferably, X′ is a halide.

In embodiments, quaternary ammonium ether salts which may be useful as decomplexing agents may include quaternaries sold by Tomah³ Products Inc. in the Q-Series of quaternary amine products. The Tomah³ quaternaries are based on the reaction of high molecular weight aliphatic tertiary amines with an alkylating agent such as methyl chloride in a diluent such as isopropyl alcohol. Preparation of such quaternary amines is known in the art.

Suitable examples of such ether amine quaternaries of formula (B) include, but are not limited to, the products Q-14-2 and Q-14-2 PG (isodecyloxypropyl dihydroxyethylmethyl ammonium chloride, where R is branched C₁₀H₂₁, X′ is chloride and x and y yield a molecular weight of about 370), Q-17-2 and Q-17-2 PG (isotridecyloxypropyl dihydroxyethylmethyl ammonium chloride, where R is branched C₁₃H₂₇, X′ is chloride and x and y yield a molecular weight of about 410), and Q-17-5 (isotridecyloxypropyl poly(5) oxyethylene methyl ammonium chloride, where R is branched C₁₃H₂₇, X′ is chloride and x and y yield a molecular weight of about 535), all available from the Tomah³ Products Inc. company.

In embodiments, quaternary amines which may be used as the quaternary ammonium ether salt include compounds such as octadecyl poly(15)oxyethylene methyl ammonium chloride (Q-18-15), 50% active octadecyl dihydroxyethyl methyl ammonium chloride (Q-18-2(50)), or other quaternaries available from the Tomah³ Products Inc. Q-Series.

The amount of polymerizable quaternary ammonium ether salt to be used as decomplexing agent for and with the polymerizable monomeric cyanoacrylate adhesive compositions comprising a complexing agent comprising BF₃, typically may depend on the amount of boron trifluoride present in the polymerizable monomeric cyanoacrylate composition, the type or types of cyanoacrylate monomers present and the desired rate of polymerization. Typically, the quaternary ammonium ether salt will be present in an amount of from about 0.001% to about 30%, preferably about 0.05% to about 30%, by weight.

Initiator or accelerator compounds may be used in conjunction with or in combination with the quaternary ammonium fluoride or quaternary ammonium ether salt decomplexing agents described. By way of example, quaternary ammonium chloride salts are desirable as initiators particularly with alkyl ester cyanoacrylate monomers. The initiator or accelerator will be used in sufficient amount to provide the desired initiation or acceleration of polymerization of the cyanoacrylate monomer(s).

The initiator or accelerator may be in the form of a solid, such as a powder or a solid film, or in the form of a liquid, such as a viscous or paste-like material. The initiator or accelerator may also include a variety of additives, such as surfactants or emulsifiers. Preferably, the initiator or accelerator is soluble in the monomer composition, and/or comprises or is accompanied by at least one surfactant which, in embodiments, helps the initiator or accelerator co-elute with the monomer composition. In embodiments, the surfactant may help disperse the initiator or accelerator in the monomer composition.

The decomplexing agent and an accelerator or initiator, when an accelerator or initiator is used, by way of example, whether a quaternary ammonium fluoride or quaternary ammonium ether salt alone, in combination with another quaternary ammonium fluoride or ether salt, or in combination with another type of initiator or accelerator, may be applied to tissue before the monomer composition, or may be applied directly to the monomer composition once the monomer composition is applied to tissue. In embodiments, the decomplexing agent and the additional initiator or accelerator, when present, may be combined with the monomer composition just prior to applying the composition to tissue.

The selection of the decomplexing agent and an initiator or accelerator, when used, may additionally affect the rate at which the polymerized monomer is absorbed by living tissue. Therefore, the most suitable decomplexing agents and initiators or accelerators are those that initiate or accelerate polymerization of the monomer at a rate suitable for medical applications while providing a polymer that is substantially absorbed in less than three years.

For purposes herein, the phrase “suitable for medical application(s)” means that the polymerization of the monomer occurs in less than 5 minutes or less than 3 minutes, preferably in less than 2.5 minutes, more preferably in less than 1 minute, and often in less than 45 seconds. Of course, the desired polymerization time can vary for different compositions and/or uses. Preferably, where absorbability is desired, a suitable initiator or accelerator and a suitable monomer are selected to provide a polymer that is substantially absorbed by a living organism in 2-24 months, such as 3-18 months or 6-12 months after application of the adhesive to living tissue.

Suitable additional initiators are known in the art and are described, for example, in U.S. Pat. Nos. 5,928,611 and 6,620,846, both incorporated herein by reference in their entireties, and U.S. Patent Application No. 2002/0037310, also incorporated herein by reference in its entirety. Quaternary ammonium chloride and bromide salts useful as polymerization initiators are particularly suitable. By way of example, quaternary ammonium salts such as domiphen bromide, butyrylcholine chloride, benzalkonium bromide, acetyl choline chloride, among others, may be used.

Benzalkonium or benzyltrialkyl ammonium halides such as benzyltrialkyl ammonium chloride may be used in addition to one or more quaternary ammonium fluoride salts or one or more quaternary ammonium ether salts. When used, the benzalkonium halide may be benzalkonium halide in its unpurified state, which comprises a mixture of varying chain-length compounds, or it can be any suitable purified compound including those having a chain length of from about 12 to about 18 carbon atoms, including but not limited to C₁₂, C₁₃, C₁₄, C₁₅, C₁₆, C₁₇, and C₁₈ compounds. By way of example, the additional initiator may be a quaternary ammonium chloride salt such as benzyltrialkyl ammonium chloride (BTAC).

Other initiators or accelerators may also be selected by one of ordinary skill in the art without undue experimentation. Such suitable initiators or accelerators may include, but are not limited to, detergent compositions; surfactants: e.g., nonionic surfactants such as polysorbate 20 (e.g., Tween 20™ from ICI Americas), polysorbate 80 (e.g., Tween 80™ from ICI Americas) and poloxamers, cationic surfactants such as tetrabutylammonium bromide, anionic surfactants such as sodium tetradecyl sulfate, and amphoteric or zwitterionic surfactants such as dodecyldimethyl(3-sulfopropyl)ammonium hydroxide, inner salt; amines, imines and amides, such as imidazole, arginine and povidine; phosphines, phosphites and phosphonium salts, such as triphenylphosphine and triethyl phosphite; alcohols such as ethylene glycol, methyl gallate; tannins; inorganic bases and salts, such as sodium bisulfite, calcium sulfate and sodium silicate; sulfur compounds such as thiourea and polysulfides; polymeric cyclic ethers such as monensin, nonactin, crown ethers, calixarenes and polymeric-epoxides; cyclic and acyclic carbonates, such as diethyl carbonate; phase transfer catalysts such as Aliquat 336; organometallics such as cobalt naphthenate and manganese acetylacetonate; and radical initiators or accelerators and radicals, such as di-t-butyl peroxide and azobisisobutyronitrile.

In embodiments, mixtures of two or more, such as three, four, or more, initiators or accelerators can be used with one or more quaternary ammonium fluoride salts and/or one or more quaternary ammonium ether salts. A combination of multiple initiators or accelerators may be beneficial, for example, to tailor the initiator of the polymerizable monomer species. For example, where a blend of monomers is used, a blend of initiators may provide superior results to a single initiator. For example, the blend of initiators can provide one initiator that preferentially initiates one monomer, and a second initiator that preferentially initiates the other-monomer, or can provide initiation rates to help ensure that both monomer species are initiated at equivalent, or desired non-equivalent, rates. In this manner, a blend of initiators can help minimize the amount of initiator necessary. Furthermore, a blend of initiators may enhance the polymerization reaction kinetics.

In embodiments, the cyanoacrylate adhesive composition may be applied by any means known to those of skill in the art. By way of example, any suitable applicator may be used to apply the adhesive composition to a substrate. For example, the applicator may include an applicator body, which is formed generally in the shape of a tube having a closed end, an open end, and a hollow interior lumen, which holds a crushable or frangible ampoule. The applicator and its related packaging may be designed as a single-use applicator or as a multi-use applicator. Suitable multi-use applicators are disclosed, for example, in U.S. Pat. No. 6,802,416 issued Oct. 12, 2004, the entire disclosure of which is incorporated herein by reference.

In embodiments of the invention, the applicator may comprise elements other than an applicator body and an ampoule. For example, an applicator tip may be provided on the open end of the applicator. The applicator tip material may be porous, absorbent, or adsorbent in nature to enhance and facilitate application of the composition within the ampoule. Suitable designs for applicators and applicator tips that may be used according to the present invention are disclosed in, for example, U.S. Pat. Nos. 5,928,611, 6,428,233, 6,425,704, 6,455,064, and 6,372,313, the entire disclosures of which are incorporated herein by reference.

In embodiments of the present invention, an applicator may contain the decomplexing agent and an initiator or accelerator, when used, on a surface portion of the applicator or applicator tip, or on the entire surface of the applicator tip, including the interior and the exterior of the tip. When the decomplexing agent and initiator or accelerator, when used, is contained in or on an applicator tip, the decomplexing agent and initiator or accelerator, when used, may be applied to the surface of the applicator tip or may be impregnated or incorporated into the matrix or internal portions of the applicator tip, depending on the use. Additionally, the decomplexing agent and initiator or accelerator, when used, may be incorporated into the applicator tip, for example, during the fabrication of the tip.

In other embodiments, an initiator may be coated on an interior surface of the applicator body and/or on an exterior surface of an ampoule or other container disposed within the applicator body, may be placed in the applicator body in the form of a second frangible vial or ampoule and/or may be otherwise contained within the applicator body, so long as a non-contacting relationship between the polymerizable monomer composition and the initiator is maintained until use of the adhesive.

The viscosity of the polymerizable cyanoacrylate monomer or monomers and/or the monomer composition may be controlled by the addition of a thickening agent or component. The thickening agents may be selected from among known thickeners, including, but not limited to, poly(2-ethylhexyl methacrylate), poly(2-ethylhexyl acrylate) and cellulose acetate butyrate. Suitable thickeners further include, for example, polycyanoacrylates, polyoxalates, lactic-glycolic acid copolymers, polycaprolactone, lactic acid-caprolactone copolymers, poly(caprolactone+DL-lactide+glycolide), polyorthoesters, polyalkyl acrylates, copolymers of alkylacrylate and vinyl acetate, polyalkyl methacrylates, and copolymers of alkyl methacrylates and butadiene. Examples of alkyl methacrylates and acrylates are poly(butylmethacrylate) and poly(butylacrylate), also copolymers of various acrylate and methacrylate monomers, such as poly(butylmethacrylate-co-methylmethacrylate). Biodegradable polymer thickeners are preferred for some uses such as some surgical uses.

In embodiments, the thickening agent may be an absorbable polymer. Examples of such absorbable polymers are known in the art and may include absorbable polyesters. By way of example, the absorbable polymer may be a polymer, copolymer, or homopolymer of glycolide, lactide, caprolactone, trimethylene carbonate, and/or dioxanone such as a copolymer of caprolactone and L-lactide. It has now been discovered that preparation or synthesis of absorbable polymers such as absorbable polyesters with the use of a certain catalyst results in a thickening agent which provides desirable viscosity parameters for polymerizable cyanoacrylate monomer adhesive compositions. In particular, it has been discovered that use of a boron trifluoride compound or complex as the catalyst results in an absorbable polyester polymer or copolymer with improved thickening characteristics in cyanoacrylate monomeric compositions. A variety of catalysts, such as Sn(Oct)₂ and SnCl₂ are known for the preparation of absorbable polymers or copolymers such as caprolactone-lactide copolymers. Absorbable polymers or copolymers prepared or synthesized with Sn(Oct)₂ or SnCl₂, may contain residual amount of these catalysts which may affect the stability of the cyanoacrylate composition when used as a thickening agent. However, the use of the BF₃ as catalyst for synthesis of the polymer overcomes the stability problem. In addition, the selection of a BF₃ catalyst provides superior viscosity when the absorbable polymer or copolymer prepared by use of the BF₃ catalyst is utilized as a thickening agent in a polymerizable cyanoacrylate monomeric composition.

Boron compounds known in the art such as boron trifluoride, boron trifluoride diethyl etherate or other boron trifluoride complexes may be used as the catalyst. In an embodiment, for example, the boron compound or complex may be boron trifluoride diethyl etherate and the absorbable copolymer prepared may be poly(caprolactone-co-L-lactide).

A high viscosity absorbable cyanoacrylate composition may be provided by mixing polymerizable cyanoacrylate monomer or monomers and an absorbable polymer or copolymer catalyzed with BF₃ catalyst that does not impair the stability of the final product as may polymer or copolymer catalyzed by Sn(Oct)₂ or SnCl₂. The catalyst will also have stabilizing properties that will enhance the stability and/or shelf life of the final composition.

By way of example, a mixture of 2-octylcyanoacrylate and butyl lactoylcyanoacrylate may be mixed with a copolymer of L-lactide and ε-caprolactone catalyzed or polymerized with boron trifluoride. The boron trifluoride is thus used as an effective stabilizer for polymerizable monomeric cyanoacrylates and also as an effective catalyst for the making of an absorbable polymer suitable as a thickening agent. The subsequent mixture will have higher viscosity and extended shelf life.

Preferably, the thickening agent is soluble in a monomer composition at room temperature (i.e., 20-25° C.) so that it may be added to the monomer composition without excessive heating of the monomer composition and remain uniformly combined in the composition.

The amount of thickening agent that is added to the monomer composition depends upon the molecular weight of the thickening agent. The thickening agent preferably comprises from about 0.5 to about 25.0% by weight of the adhesive composition. In preferred embodiments, the thickening agent comprises from about 1.0 to about 10.0%, more preferably about 1.0 to about 5.0%, of the adhesive composition. In embodiments, the thickening agent has a high molecular weight, preferably at least 100,000, or at least 500,000 or at least 1,000,000. The thickening agent is selected such that it is compatible with the monomer (i.e., does not adversely affect polymerization, bond strength, core properties, or shelf-life). The amount of thickening agent to be used can be determined by one of ordinary skill in the art using known techniques without undue experimentation.

In embodiments, the adhesive composition has a viscosity of about 20-10,000 centipoise, preferably 30-1,000 centipoise, as measured with a Brookfield Viscometer at 25° C.

Other optional components may be present in the polymerizable cyanoacrylate compositions including plasticizers, colorants, preservatives, heat dissipating agents, additional stabilizing agents and the like. Typically, these components will be used in amount of up to about 25, more preferably up to about 10, for example, up to about 5 weight %, based on a total weight of the composition.

Preservatives useful in compositions of this invention may be anti-microbial agents. In embodiments, a preservative may be selected from among preservatives including, but not limited to, parabens and cresols. For example, suitable parabens include, but are not limited to, alkyl parabens and salts thereof, such as methylparaben, methylparaben sodium, ethylparaben, propylparaben, propylparaben sodium, butylparaben, and the like. Suitable cresols include, but are not limited to, cresol, chlorocresol, and the like. The preservative may also be selected from other known agents including, but not limited to, hydroquinone, pyrocatechol, resorcinol, 4-n-hexyl resorcinol, captan (i.e., 3a,4,7,7a-tetrahydro-2-((trichloromethyl)thio)-1H-isoindole-1,3(2H)-dione), benzoic acid, benzyl alcohol, chlorobutanol, dehydroacetic acid, o-phenylphenol, phenol, phenylethyl alcohol, potassium benzoate, potassium sorbate, sodium benzoate, sodium dehydroacetate, sodium propionate, sorbic acid, thimerosal, thymol, phenylmercuric compounds such as phenylmercuric borate, phenylmercuric nitrate and phenylmercuric acetate, formaldehyde, and formaldehyde generators such as the preservatives Germall II® and Germall 115® (imidazolidinyl urea, available from Sutton Laboratories, Charthan, N.J.). Other suitable preservatives are disclosed in U.S. Pat. No. 6,579,469, the entire disclosure of which is hereby incorporated by reference. In embodiments, mixtures of two or more preservatives may also be used.

Monomer compositions of the invention may also include a heat dissipating agent. Heat dissipating agents include liquids or solids that may be soluble or insoluble in the monomer. The liquids may be volatile and may evaporate during polymerization, thereby releasing heat from the composition. Suitable heat dissipating agents may be found in U.S. Pat. No. 6,010,714 to Leung et al., the entire disclosure of which is incorporated herein.

The composition may also optionally include at least one plasticizing agent that imparts flexibility to the polymer formed from the monomer. The plasticizing agent preferably contains little or no moisture and should not significantly affect the stability or polymerization of the monomer. Such plasticizers are useful in polymerized compositions to be used for closure or covering of wounds, incisions, abrasions, sores or other applications where flexibility of the adhesive is desirable.

Examples of suitable plasticizers include acetyl tributyl citrate, dimethyl sebacate, dibutyl sebacate, triethyl phosphate, tri(2-ethylhexyl)phosphate, tri(p-cresyl)phosphate, glyceryl triacetate, glyceryl tributyrate, diethyl sebacate, dioctyl adipate, isopropyl myristate, butyl stearate, lauric acid, trioctyl trimellitate, dioctyl glutarate, polydimethylsiloxane, and mixtures thereof. Preferred plasticizers may include tributyl citrate, acetyl tributyl citrate or dibutyl sebacate. In embodiments, suitable plasticizers include polymeric plasticizers, such as polyethylene glycol (PEG) esters and capped PEG esters or ethers, polyester glutarates and polyester adipates.

The addition of plasticizing agents in amounts ranging from about 0.5 wt. % to about 25 wt. %, or from about 1 wt. % to about 20 wt. %, or from about 3 wt. % to about 15 wt. % or from about 5 wt. % to about 7 wt. % provides increased elongation and toughness of the polymerized monomer over polymerized monomers not having plasticizing agents.

The composition may also optionally include at least one thixotropic agent. Suitable thixotropic agents are known to the skilled artisan and include, but are not limited to, silica gels such as those treated with a silyl isocyanate. Examples of suitable thixotropic agents are disclosed in, for example, U.S. Pat. No. 4,720,513, the disclosure of which is hereby incorporated in its entirety.

The composition may also optionally include at least one natural or synthetic rubber to impart impact resistance, which is preferable especially for industrial compositions of the present invention. Suitable rubbers are known to the skilled artisan. Such rubbers include, but are not limited to, dienes, styrenes, acrylonitriles, and mixtures thereof. Examples of suitable rubbers are disclosed in, for example, U.S. Pat. Nos. 4,313,865 and 4,560,723, the disclosures of which are hereby incorporated in their entireties.

Medical compositions of the present invention may also include at least one biocompatible agent effective to reduce active formaldehyde concentration levels produced during in vivo biodegradation of the polymer (also referred to herein as “formaldehyde concentration reducing agents”). Preferably, this component is a formaldehyde scavenger compound. Examples of useful formaldehyde scavenger compounds include sulfites; bisulfites; and mixtures of sulfites and bisulfites, among others. Useful additional examples of formaldehyde scavenger compounds and methods for their implementation may be found U.S. Pat. Nos. 5,328,687, 5,514,371, 5,514,372, 5,575,997, 5,582,834 and 5,624,669, all to Leung et al., which are hereby incorporated herein by reference in their entireties. A preferred formaldehyde scavenger is sodium bisulfite.

In preferred embodiments, the formaldehyde concentration reducing agent is added in an effective amount to the cyanoacrylate. The “effective amount” is that amount sufficient to reduce the amount of formaldehyde generated during subsequent in vivo biodegradation of the polymerized cyanoacrylate. This amount will depend on the type of active formaldehyde concentration reducing agent, and can be readily determined without undue experimentation by those skilled in the art.

The formaldehyde concentration reducing agent may be used in either free form or in microencapsulated form. When microencapsulated, the formaldehyde concentration reducing agent is released from the microcapsule continuously over a period of time during the in vivo biodegradation of the cyanoacrylate polymer.

The microencapsulated form of the formaldehyde concentration reducing agent is preferred because this embodiment prevents or substantially reduces polymerization of the cyanoacrylate monomer by the formaldehyde concentration reducing agent, which increases shelf-life and facilitates handling of the monomer composition during use. Microencapsulation techniques are disclosed in U.S. Pat. No. 6,512,023, incorporated herein by reference in its entirety.

By way of example, in one embodiment, the cyanoacrylate adhesive composition comprises about 75% 2-octylcyanoacrylate, about 25% butyl lactoylcyanoacrylate, about 50 ppm boron trifluoride, less than about 70 ppm hydroquinone, about 1600 ppm butylated hydroxyanisole, about 110 ppm p-methoxyphenol, about 5.0 ppm sulfuric acid, about 15.0 ppm sulfur dioxide, and about 103.0 ppm acetic acid. The cyanoacrylate adhesive composition may be used, for example, with about 2000 ppm of a quaternary ammonium fluoride salt or about 5% of a quaternary ammonium ether salt as decomplexing agent, and, optionally, about 1125 ppm of a quaternary ammonium chloride salt as an additional initiator.

Having generally described embodiments of the invention, a further understanding can be obtained by reference to certain specific examples which are provided herein for purposes of illustration only and are not intended to be limiting unless otherwise specified.

### EXAMPLES

### Example 1

### Effect of BF₃ on Stability and Reactivity of a Monomeric Cyanoacrylate Formulation

To evaluate the effect of the use of BF₃ as a stabilizer/complexing agent on the stability of an absorbable cyanoacrylate adhesive formulation comprising one or more cyanoacrylate monomers, monomeric cyanoacrylate compositions with 0, 50, 80, or 120 ppm of BF₃ were prepared and measured for viscosity at various time points at 80° C. The monomeric cyanoacrylate composition included 75%/25% 2-octylcyanoacrylate/butyl lactoylcyanoacrylate, less than about 70 ppm hydroquinone, about 1600 ppm butylated hydroxyanisole, about 110 ppm p-methoxyphenol, about 20 ppm sulfuric acid, about 11 ppm sulfur dioxide and about 106 ppm acetic acid.

The viscosity was measured using a Brookfield Cone/Plate Viscometer with spindle CP-40 by known methods at 0, 6, 12 and 18 days. At 0 and 6 days, all four formulations had similar low viscosity as shown in Table 1. At 12 days, the viscosity levels for all samples increased slightly, to approximate a 2-fold increase for the formulations with BF₃ and more than a 3-fold increase for the formulation without BF₃. At 18 days, the viscosity of the formulations with BF₃ was at 36-54 cps, whereas the viscosity of the formulation without BF₃ was more than 1,000 cps.

TABLE 1

| | |
|---|---|
| Formulation | Viscosity (cps) |

| Description | T(0) | T(6) | T(12) | T(18) |
|---|---|---|---|---|
| 0 ppm BF₃ | 7.5 | 9.0 | 30 | >1000 |
| 50 ppm BF₃ | 7.0 | 8.0 | 14 | 46 |
| 80 ppm BF₃ | 7.0 | 7.9 | 13 | 36 |
| 120 ppm BF₃ | 7.0 | 8.0 | 14 | 54 |

To evaluate the effect of BF₃ on reactivity, a constant amount of a polymerization initiator (benzyltrialkyl ammonium chloride (BTAC)) was added to the BF₃ formulations for a gel time study. The gel time study was conducted with the method as stated. The amount of 1.850 g±0.050 g of the cyanoacrylate formulation was added into a 7 mL glass scintillation vial with stir bar. The vial was placed on a stir plate and the speed of the stirring was set such that a vortex was reached half way from the surface of the liquid to the bottom of the vial. 200 uL of the appropriate concentration of initiator solution was added into the vial. Timing was started immediately with a stop watch when the initiator solution was added. The timing was stopped when the stir bar stopped, the vial fell over, and/or the liquid reacted violently. The above procedure was conducted three times for three data sets.

As shown in Table 2, the gel time for all formulations with BF₃ was more than two minutes compared to 5 seconds for the formulation without BF₃. Additionally, the gel time increased at higher BF₃ concentration. This indicates that the addition of BF₃ increases the stability but decreases the reactivity of the monomeric cyanoacrylate formulation.

TABLE 2

| Formulation | Initiator | Gel Time T(0) |
|---|---|---|
| 0 ppm BF₃ | .225% BTAC in Acetone | 5 seconds |
| 50 ppm BF₃ | .225% BTAC in Acetone | 2 minutes |
| 80 ppm BF₃ | .225% BTAC in Acetone | 13 minutes |
| 120 ppm BF₃ | .225% BTAC in Acetone | >1.5 hours |

### Example 2

### Effect of Fluoride Salt of Quaternary Amine Decomplexing Agents on Stability and Reactivity of a Cyanoacrylate Formulation with BF₃

To address the reduced reactivity of the BF₃ formulations, the fluoride or ether salt of a quaternary amine was employed as a decomplexing agent for the formulation. Results of the reactivity study of the fluoride salts are summarized in Tables 3-5.

Tetrabutyl ammonium fluoride (TBAF) or benzyltrimethyl ammonium fluoride (BTMAF) was combined with a constant amount of BTAC and the BF₃ formulations described in Example 1. As shown in Tables 3-5, the gel time of the BF₃ formulations was significantly shorter when both BTAC and a quaternary ammonium fluoride salt were present. Also, both quaternary ammonium fluoride salts displayed similar effects on reactivity. Further, the gel time of the BF₃ formulations was reduced when higher concentrations of quaternary ammonium fluoride salt were used.

TABLE 3

| 50 ppm BF₃ | | | T(0) | T(6) | T(12) | T(18) |
|---|---|---|---|---|---|---|
| Solvent | Initiator | Agent | Gel Time (s) | | | |
| 50/50 | .1125% | 1000 ppm TBAF | 66 | 99 | 122 | 74 |
| Acetone/ | BTAC | 1500 ppm TBAF | 22 | 42 | 37 | 7 |
| MeOH | | 2500 ppm TBAF | 5 | 11 | 9 | 2 |
| | | 625 ppm BTMAF | 66 | 142 | 156 | 62 |
| | | 1000 ppm BTMAF | 24 | 52 | 38 | 6 |
| | | 1750 ppm BTMAF | 5 | 14 | 8 | 4 |

TABLE 4

| 80 ppm BF₃ | | | T(0) | T(6) | T(12) | T(18) |
|---|---|---|---|---|---|---|
| Solvent | Initiator | Agent | Gel Time (s) | | | |
| 50/50 | .1125% | 1750 ppm TBAF | 38 | 158 | 103 | 30 |
| Acetone/ | BTAC | 2500 ppm TBAF | 14 | 41 | 29 | 5 |
| MeOH | | 4000 ppm TBAF | 3 | 8 | 6 | 4 |
| | | 1250 ppm BTMAF | 54 | 139 | 202 | 34 |
| | | 1500 ppm BTMAF | 27 | 74 | 54 | 9 |
| | | 2500 ppm BTMAF | 5 | 18 | 11 | 7 |

TABLE 5

| 120 ppm BF₃ | | | T(0) | T(6) | T(12) | T(18) |
|---|---|---|---|---|---|---|
| Solvent | Initiator | Agent | Gel Time (s) | | | |
| 50/50 | .1125% | 3500 ppm TBAF | 40 | 36 | 33 | 5 |
| Acetone/ | BTAC | 4000 ppm TBAF | 7 | 11 | 12 | 3 |
| MeOH | | 5000 ppm TBAF | 6 | 6 | 7 | 2 |
| | | 2000 ppm BTMAF | 56 | 47 | 61 | 7 |
| | | 2500 ppm BTMAF | 23 | 44 | 36 | 2 |
| | | 4000 ppm BTMAF | 5 | 8 | 6 | 1 |

### Example 3

Additional tests were conducted using the same cyanoacrylate formulation as in Examples 1 and 2. The amounts of TBAF and BTMAF varied in the gel time study. The results of a gel time study using the cyanoacrylate formulation with BF₃ and BTAC are shown in Table 6. The results of a gel time study with varying amounts of fluoride salt and BF₃ are shown in Table 7:

TABLE 6

| Formulation | Initiator | Gel Time T(0) |
|---|---|---|
| 0 ppm BF₃ | .225% BTAC in Acetone | 6 seconds |
| 50 ppm BF₃ | .225% BTAC in Acetone | 1.5 minutes |
| 80 ppm BF₃ | .225% BTAC in Acetone | 10 minutes |
| 120 ppm BF₃ | .225% BTAC in Acetone | >3 hours |

TABLE 7
Gel Times

Gel Time

| | | | | |
|---|---|---|---|---|
| Agent | T(0) | T(6) | T(12) | T(15) |

50 ppm BF₃

| | | | | |
|---|---|---|---|---|
| 4000 ppm TBAF | 3 | 5 | 2 | 2 |
| 2500 ppm BTMAF | 5 | 10 | 3 | 2 |

80 ppm BF₃

| | | | | |
|---|---|---|---|---|
| 5000 ppm TBAF | 4 | 6 | 3 | 3 |
| 4000 ppm BTMAF | 3 | 5 | 3 | 3 |

120 ppm BF₃

| | | | | |
|---|---|---|---|---|
| 7500 ppm TBAF | 6 | 6 | 4 | 3 |
| 5000 ppm BTMAF | 5 | 5 | 4 | 3 |

### Example 4

### Effect of Ether Salt of Quaternary Amine Decomplexing Agents on Stability and Reactivity of a Cyanoacrylate Formulation with BF₃

To examine the effect of quaternary ammonium ether salts, the BF₃ formulations described in Example 1 were used with a quaternary ammonium ether salt, Q-18-15, from Tomah³ Products, Inc., (octadecyl poly(15)oxyethylene methyl ammonium chloride) or octadecyl poly(15)oxyethylene methyl ammonium chloride and BTAC. The quaternary ammonium ether salt Q-18-15 was used in amounts of 0.5%, 1%, 5%, 10% and 20% by weight in methanol solution. These compositions were applied via prototype applicators in the gel time experiment. Q-18-15 was used by itself and in combination with BTAC and/or TBAF. The results in Table 8 show that the quaternary ammonium ether salt alone was able to adequately promote initiation of the polymerization of the BF₃ formulations. The set time was further reduced when both the quaternary ammonium ether salt and BTAC were used in combination.

TABLE 8

| | | | | | | 3500 ppm TBAF & | |
|---|---|---|---|---|---|---|---|
| Q-18-15 only | | 3500 ppm TBAF | | 1125 ppm BTAC | | 1125 ppm BTAC | |
| set time (s) | average (s) | set time (s) | average (s) | set time (s) | average (s) | set time (s) | average (s) |

20% Q-18-15

| | | | | | | | |
|---|---|---|---|---|---|---|---|
| 25 | 20 | 30 | 26 | 9 | 13 | 22 | 18 |
| 18 | | 25 | | 18 | | 13 | |
| 15 | | 23 | | 11 | | 18 | |
| 22 | | N/A | | N/A | | N/A | |

10% Q-18-15

| | | | | | | | |
|---|---|---|---|---|---|---|---|
| 14 | 13 | 30 | 32 | 18 | 19 | 16 | 19 |
| 12 | | 27 | | 17 | | 22 | |
| 13 | | 40 | | 23 | | 19 | |

5% Q-18-15

| | | | | | | | |
|---|---|---|---|---|---|---|---|
| 17 | 16 | 35 | 32 | 14 | 17 | 40 | 45 |
| 13 | | 35 | | 19 | | 53 | |
| 18 | | 27 | | 17 | | 41 | |

1% Q-18-15

| | | | | | | | |
|---|---|---|---|---|---|---|---|
| 80 | 72 | >180 | >180 | 30 | 32 | >60 | >180 |
| 64 | | >180 | | 34 | | >180 | |
| 72 | | >180 | | 32 | | >180 | |

.5% Q-18-15

| | | | | | | | |
|---|---|---|---|---|---|---|---|
| 130 | 119 | >180 | >180 | 32 | 48 | >180 | >180 |
| 113 | | >180 | | 47 | | >180 | |
| 114 | | >180 | | 64 | | >180 | |

Taken together, the stability of the absorbable cyanoacrylate composition is improved with the addition of BF₃. Though the reactivity is reduced in the BF₃ formulations, a quaternary ammonium fluoride salt or an ether based quaternary ammonium salt decomplexing agent is able to initiate and/or accelerate the polymerization of the formulation.

### Example 5

Applicator devices were assembled and initiated with solutions containing either 4 or 5% Q-18-15 and either 1600 or 3200 ppm of BTAC in acetone using the cyanoacrylate formulation of Example 1 and 50 ppm BF₃. The same cyanoacrylate formulation containing 1600 ppm of BTAC was used as control. The formulations were applied from the devices for tests on skin block and micro gel tests before and after ethylene oxide (ETO) sterilization. The skin block and micro gel tests were conducted as follows. 0.15 g±0.050 g of the cyanoacrylate formulation via the applicator with the appropriate amount of initiator/accelerator was added into a 0.5 mL glass auto sampler vial. The vial was placed on a block against a liquid crystal sheet with adhesive backing. Timing was started immediately with a stop watch upon the addition of the appropriate amount of the formulation. When the color of liquid crystal coated sheet changed from black to bluish green, the timing was stopped. For setting time on a skin block, a designated area 1 cm² was identified and the needle thermocouple was inserted through the surface of the polyurethane block outside the 1 cm² work area, so the tip of the needle was located at the center of the 1 cm² application area. The timer was started at the same instance when the cyanoacrylate was mixed with the initiator/accelerator. The first two drops were discarded and the third drop was placed on the surface of the polyurethane block over the exposed tip of the needle, spreading the drop evenly over the 1 cm² application area and recording the time for the complete polymerization or until solidification of the monomer.

The results of gel time before and after sterilization are shown in Table 9.

TABLE 9

| Cyanoacrylate | | | Set time on | Micro gel | Set time on | Micro gel |
|---|---|---|---|---|---|---|
| (CA) | | | Skin Block (s) | time (s) | Skin Block (s) | time (s) |
| Formulation | Q-18-15 | BTAC | Pre-ETO | Pre-ETO | Post-ETO | Post-ETO |
| CA w/50 ppm | 4% | 1600 ppm | 39 | 18 | >5 minutes | 82 |
| BF₃ | 5% | 1600 ppm | 26 | 15 | >5 minutes | 67 |
| | 4% | 3200 ppm | 65 | 22 | >5 minutes | 61 |
| | 5% | 3200 ppm | 39 | 18 | >5 minutes | 54 |
| Control CA | 0% | 1600 ppm | >4 minutes | 31 | >5 minutes | 55 |

After sterilization, the formulations containing BF₃ and Q-18-15 had setting times of more than 5 minutes on the skin block test and under two minutes in micro gel tests. These setting times were slower than the pre ETO samples. The control samples did not set on the skin block before or after sterilization. The micro gel time of the control samples also increased by 24 seconds after ETO.

Two additional sets of application devices were assembled and initiated. The application devices contained cyanoacrylate formulations as described in Example 1. One set was initiated/accelerated with a solution containing 10% Q-18-15 and 3200 ppm of BTAC. The second set was initiated/accelerated with a solution containing 5% Q-18-15 and 6400 ppm of BTAC. After ETO sterilization, both sets of devices had a set time on the skin block of about 100 seconds and a micro gel time of 23 seconds. Control devices were assembled and initiated with 0.01 M of BTAC. The post ETO set time for the control was 78 seconds on the skin block and 12 seconds with the micro gel test.

Alternative Tomah³ decomplexing agents were also tested, such as Q-17-2, Q-17-5, Q-14-2, and Q-18-2 (50). This testing was performed with non sterile devices, but all four agents were able to polymerize the BF₃ formulation. The setting times on the skin block were 52, 63, 53, and 178 seconds respectively.

### Example 6

### Stability Study of Monomeric Cyanoacrylate Formulation Thickened by Poly(Caprolactone-Co-L-Lactide)

A cyanoacrylate monomer formulation was prepared as described in Example 1. The monomeric cyanoacrylate composition included 75%/25% 2-octylcyanoacrylate/butyl lactoylcyanoacrylate, less than about 70 ppm hydroquinone, about 1600 ppm butylated hydroxyanisole, about 110 ppm p-methoxyphenol, about 20 ppm sulfuric acid, about 11 ppm sulfur dioxide and about 106 ppm acetic acid.

Using the same cyanoacrylate formulation for each, three lots, Lots 1, 2 and 3 were prepared and contained a thickening agent of poly(caprolactone-co-L-lactide) synthesized with boron trifluoride diethyl etherate (BF₃) as the catalyst. Lot 4 contained poly (caprolactone-co-L-lactide) synthesized with tin (II) 2-ethylhexanoate as the catalyst. The amount of catalyst used was 0.0004 moles per 2 moles of monomer. The amount of thickening agent was 10% of the total weight.

Stability studies were conducted at 160° C. for 30 minutes. Tables 10 and 11 show the results of the stability studies before and after the temperature treatment, respectively.

TABLE 10
Viscosity Data prior to Exposure to 160° C. for 30 minutes

| | | | Viscosity (cP) | | | |
|---|---|---|---|---|---|---|
| | Lot | 1 | 2 | 3 | AVE | STDEV |
| No thickening agent | | 7.3 | 7.3 | 7.3 | 7.3 | 0.0 |
| Formulations thickened | 1 | 19.8 | 20.5 | 20.6 | 20.3 | 0.4 |
| with BF₃ catalyzed | 2 | 22.1 | 21.9 | 21.7 | 21.9 | 0.2 |
| polymer | 3 | 23.9 | 23.7 | 23.6 | 23.7 | 0.2 |
| Formulation thickened with | 4 | 165.7 | 168.1 | 162.9 | 165.6 | 2.6 |
| tin (II) 2-ethylhexanoate | | | | | | |
| catalyzed polymer | | | | | | |

TABLE 11
Viscosity Data After Exposure to 160° C. for 30 minutes

| | | | | Viscosity (cP) | | |
|---|---|---|---|---|---|---|
| | Lot | 1 | 2 | 3 | AVE | STDEV |
| No thickening agent | | 9.8 | 9.7 | 9.7 | 9.7 | 0.1 |
| Formulations thickened with | 1 | 46.4 | 47.0 | 46.0 | 46.5 | 0.5 |
| BF₃- catalyzed polymer | 2 | 42.8 | 42.7 | 44.4 | 43.3 | 1.0 |
| | 3 | 47.7 | 47.3 | 47.4 | 47.5 | 0.2 |
| Formulation thickened with | 4 | * | * | * | * | * |
| tin (II) 2-ethylhexanoate - | | | | | | |
| catalyzed polymer | | | | | | |

* No measurement was collected for this lot because the material was solid.

While the invention has been described with reference to preferred embodiments, the invention is not limited to the specific examples given, and other embodiments and modifications can be made by those skilled in the art without departing from the spirit and scope of the invention.

1. An adhesive composition comprising:
one or more polymerizable cyanoacrylate monomers,
a complexing agent for the one or more polymerizable cyanoacrylate monomers comprising boron trifluoride, and
a decomplexing agent comprising at least one quaternary ammonium fluoride salt, at least one quaternary ammonium ether salt, or mixtures thereof.

2. The adhesive composition of claim 1 wherein the quaternary ammonium fluoride salt comprises a compound according to formula A: 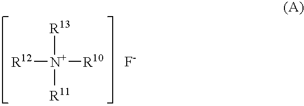
wherein R¹⁰, R¹¹, R¹² and R¹³ are each, independently, a substituted or unsubstituted straight, branched or cyclic alkyl group; a substituted or unsubstituted aromatic ring; or a substituted or unsubstituted aralkyl group, wherein the alkyl group, the aromatic ring or the aralkyl group optionally may include a heteroatom S or O.

3. The adhesive composition of claim 1 wherein the quaternary ammonium ether salt comprises a compound according to formula B: 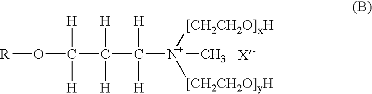
where R is a straight or branched alkyl group of from about 2 to about 20 carbon atoms, x and y represent the number of repeating units and independently are integers of from 1 to about 10, and X′ is chloride, bromide, iodide, fluoride, sulfate, hydrogen sulfate, sulfite, hydrogen sulfite, bisulfate, bisulfite, or hydroxide.

4. The adhesive composition of claim 1 further comprising a polymerization initiator comprising a quaternary ammonium chloride salt.

5. The adhesive composition of claim 1 wherein the one or more polymerizable cyanoacrylate monomers are 2-octyl cyanoacrylate and butyl lactoyl cyanoacrylate.

6. The adhesive composition of claim 1 further comprising a thickening agent comprising an absorbable polyester catalyzed by a boron trifluoride catalyst.

7. An adhesive composition comprising:
a first monomer species comprising an alkyl ester cyanoacrylate having the formula 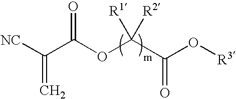
wherein R^{1′} and R^{2′} are, independently, H, a straight, branched or cyclic alkyl, or are combined together in a cyclic alkyl group, R^{3′} is a straight, branched or cyclic alkyl group, and m is 1-8;
a second monomer species different from the first monomer species;
a complexing agent for at least the first monomer species comprising boron trifluoride;
a decomplexing agent comprising at least one quaternary ammonium fluoride salt, at least one quaternary ammonium ether salt, or mixtures thereof; and
a polymerization initiator comprising at least one quaternary ammonium chloride salt.

8. The adhesive composition of claim 7 wherein the second monomer species is an alkyl α-cyanoacrylate.

9. The adhesive composition of claim 8 wherein the alkyl α-cyanoacrylate is octyl 2-cyanoacrylate.

10. The adhesive composition of claim 7 further comprising a thickening agent comprising an absorbable polyester catalyzed by a boron trifluoride catalyst.

11. The adhesive composition of claim 7 wherein the quaternary ammonium fluoride salt comprises a compound according to formula A: 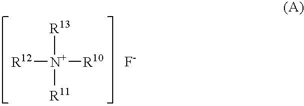
wherein R¹⁰, R¹¹, R¹² and R¹³ are each, independently, a substituted or unsubstituted straight, branched or cyclic alkyl group; a substituted or unsubstituted aromatic ring; or a substituted or unsubstituted aralkyl group, wherein the alkyl group, the aromatic ring or the aralkyl group optionally may include a heteroatom S or O.

12. The adhesive composition of claim 7 wherein the quaternary ammonium ether salt comprises a compound according to formula B: 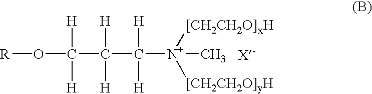
where R is a straight or branched alkyl group of from about 2 to about 20 carbon atoms, x and y represent the number of repeating units and independently are integers of from 1 to about 10, and X′ is chloride, bromide, iodide, fluoride, sulfate, hydrogen sulfate, sulfite, hydrogen sulfite, bisulfate, bisulfite, or hydroxide.

13. The adhesive composition of claim 7 further comprising a plasticizer.

14. An adhesive composition comprising:
one or more polymerizable cyanoacrylate monomers,
at least one anionic stabilizer,
at least one free radical stabilizer, and
at least one thickening agent comprising an absorbable polyester catalyzed by a boron trifluoride catalyst.

15. A method of treating living tissue, comprising:
applying to living tissue a biocompatible adhesive composition comprising one or more polymerizable cyanoacrylate monomers and a complexing agent for the one or more polymerizable cyanoacrylate monomers comprising boron trifluoride, wherein the biocompatible adhesive composition is applied in conjunction with a decomplexing agent comprising at least one quaternary ammonium fluoride salt, at least one quaternary ammonium ether salt, or mixtures thereof.

16. The method of claim 15 wherein the biocompatible adhesive composition further comprises a polymerization initiator comprising a quaternary ammonium chloride salt.

17. The method of claim 15 wherein the living tissue is internal living tissue.

18. The method of claim 15 wherein the one or more polymerizable cyanoacrylate monomers are 2-octyl cyanoacrylate and butyl lactoyl cyanoacrylate.

19. The method of claim 15 wherein the biocompatible adhesive composition is sterilized by dry heat sterilization prior to being applied to living tissue.

20. The method of claim 15 wherein the biocompatible adhesive composition further comprises a thickening agent comprising an absorbable polyester catalyzed by a boron trifluoride catalyst.
